(19) United States
(12) Patent Application Publication
(10) Pub. No.: US 2020/0143929 A1
(43) Pub. Date: May 7, 2020

(54) INTEGRATED WELLNESS SYSTEM FOR STATIONARY UNITS

(71) Applicant: Coddle Inc., Newark, CA (US)

(72) Inventors: Sujeewa Sean Pathiratne, San Jose, CA (US); Vikram Tuli, San Jose, CA (US)

(21) Appl. No.: 16/672,317

(22) Filed: Nov. 1, 2019

Related U.S. Application Data
(60) Provisional application No. 62/755,970, filed on Nov. 5, 2018.

(51) Int. Cl. G16H 20/30; G16H 40/67; G16H 50/30; G16H 80/00; A47C 1/024; A61B 5/00; G06N 20/10

(52) U.S. Cl. CPC G16H 20/30; G16H 40/67; G16H 50/30; G06N 20/10; A47C 1/0242; A61B 5/0006; G16H 80/00

(57) ABSTRACT

An integrated wellness system uses biometric sensor data received from an integrated stationary unit to generate user-specific wellness mappings and to automatically adjust one or more components of the stationary unit. Sensors in the integrated stationary unit transmit biometric sensor data to an integrated online system responsive to detecting that a user is interacting with the integrated stationary unit. Modules of the online system input the received sensor data into a trained configuration model that outputs a recommended optimal configuration of the integrated stationary unit for the user and instruct the integrated stationary unit to configure one or more components according to the recommended configuration. In some embodiments, the online system instructs the integrated stationary unit to further modify one or more components based on user input through a wellness application executing on a client device.

### CROSS REFERENCE TO RELATED APPLICATIONS

This application claims a benefit of U.S. Provisional Application No. 62/755,970, filed Nov. 5, 2018, which is incorporated by reference in its entirety.

### TECHNICAL FIELD

The subject matter described generally relates to the field of smart appliances, and in particular, to an integrated system that uses biometric sensor data from a stationary unit to generate user-specific wellness mappings and to automatically make physical configuration adjustments of the stationary unit.

### BACKGROUND

Current stationary units, such as couches and chairs, are limited in design and functionality to accommodate only basic and generic ergonomic needs of users. Users who wish to adjust one or more components of these units must do so manually and independently and use trial and error to find a comfortable position. However, while such manual adjustments may result in user comfort, the user-specified configuration might not be ergonomically optimal and may ultimately cause more discomfort to the user in the long term. Even if the user manually configures the unit to an ergonomically optimal position, the adjustments are not stored such that if the unit is returned to its initial configuration, the user will need to make the same manual adjustments each time he or she wishes to return to the ergonomically optimal position. Nor does the unit provide any mechanism for gathering evidence of user comfort and discomfort in a meaningful manner or use gathered data to provide feedback to the user about managing physical mechanics and increasing overall wellness.

### BRIEF DESCRIPTION OF THE DRAWINGS

FIG. 1 is a block diagram illustrating an integrated wellness system, according to one embodiment.

FIG. 2 is a block diagram illustrating a system architecture of an integrated online system, according to one embodiment.

FIG. 3 illustrates an exemplary integrated stationary unit with one or more biometric sensors, according to one embodiment.

FIG. 4 is an interaction diagram illustrating a method for training a configuration model to output user-specific configurations of the integrated stationary unit and feedback on postures and corrective measures to users of the integrated wellness system, according to one embodiment, according to one embodiment.

FIG. 5 is an interaction diagram illustrating a method for using the trained configuration model to generate a user-specific configuration of the integrated stationary unit, according to one embodiment.

FIGS. 6A-6E illustrate example preset configurations of an integrated stationary unit, according to one embodiment.

FIG. 7 illustrates a computer suitable for use in the integrated wellness system of FIG. 1, according to one embodiment.

### DETAILED DESCRIPTION

The Figures (FIGS.) and the following description describe certain embodiments by way of illustration only. One skilled in the art will readily recognize from the following description that alternative embodiments of the structures and methods may be employed without departing from the principles described. Reference will now be made to several embodiments, examples of which are illustrated in the accompanying figures. It is noted that wherever practicable similar or like reference numbers are used in the figures to indicate similar or like functionality.

### Overview and Benefits

Disclosed by way of embodiment is an integrated wellness system that is structured to receive biometric sensor data from an integrated stationary unit. The data may be processed to generate user-specific wellness mappings and to automatically generate signals to adjust one or more mechanical and/or micro-electro mechanical systems (MEMS) components of the stationary unit. An example configuration may include a client device (e.g., a computing or other processing device) executing an application associated with the integrated wellness system. A user, through the client device, can provide input through the application to view or modify wellness mappings and recommended configurations. The example configuration also may include an integrated stationary unit with a plurality of biometric sensors positioned at designated locations throughout the integrated stationary unit. An integrated online system also may be included that uses a configuration model to generate recommended configurations of the integrated stationary unit based on received biometric sensor data and automatically send adjustment instructions to the integrated stationary unit.

By incorporating components into a single, comprehensive system, the integrated wellness system combines hardware and software in a manner that morphs the physical attributes of the stationary unit conducive to a user's comfort and reduces the risk of injury. The configuration model feeds the integrated stationary unit with real-time recommendations for physical configuration adjustments based on data gathered by the biometric sensors. The model additionally uses the received biometric sensor data to generate and provide to the user behavioral heuristics to improve user wellness. Finally, by storing sensor data and recommended configurations associated with the user, the system can automatically adjust one or more components of the integrated stationary unit responsive to determining that the user is interacting with the integrated stationary unit, thus obviating the need for repeated manual adjustments by the user.

### Example Systems

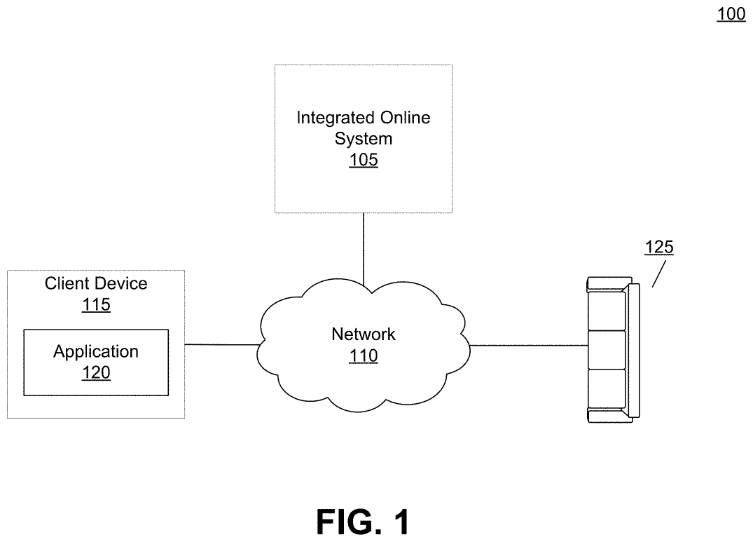

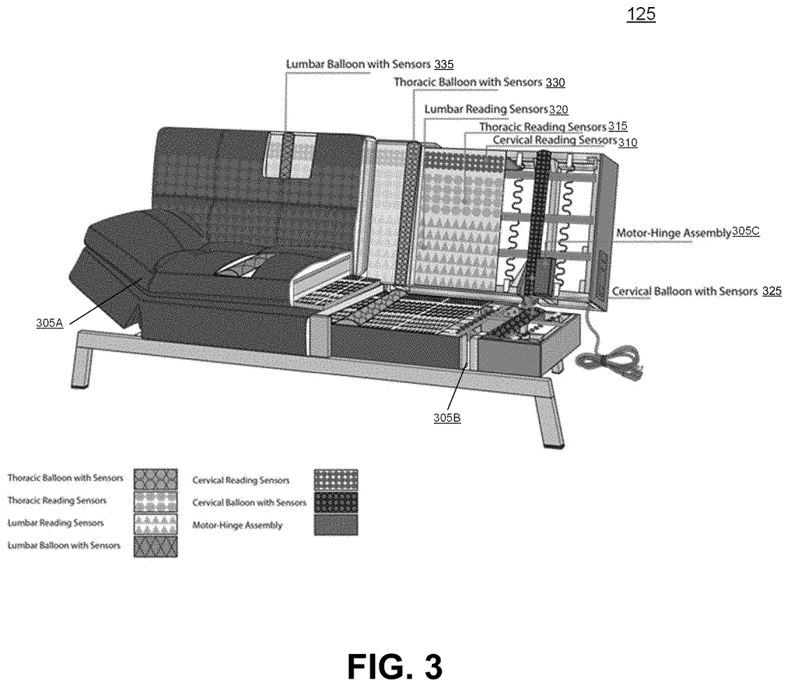

Figure (“FIG.”) 1 illustrates one embodiment of an integrated wellness system 100 that uses user-specific wellness mappings generated based on received sensor data to adjust the physical configuration of an integrated stationary unit. In the embodiment shown in FIG. 1, the integrated wellness system 100 includes an integrated online system 105 in communication through a network 110 with a client device 115 executing a wellness application 120, and an integrated stationary unit 125 with one or more biometric sensors 130 (shown in FIG. 3). In other embodiments, the integrated wellness system 100 contains different and/or additional elements. In addition, the functions may be distributed among the elements in a different manner than described.

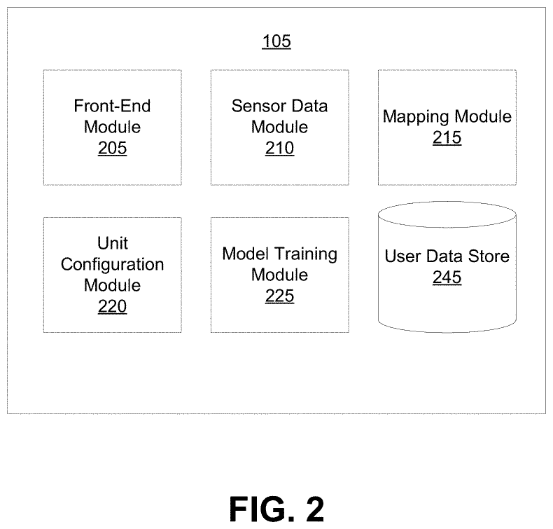

The integrated online system 105 receives sensor data from the biometric sensors 130 in the integrated stationary unit 125 and uses the received data to generate user-specific wellness mappings. Machine learning techniques may be used to generate a configuration model that uses the sensor data and generated mappings to compute user-specific recommended configurations of one or more components of the integrated stationary unit 125. These recommended configurations may further be adjusted based on user input through the client device 115. Modules of the integrated online system 105 further generate one or more preset configurations of the integrated stationary unit 125 and corresponding wellness indices based on an associated user activity, such as reading, watching television, or working on a laptop or other device. As discussed in more detail below with respect to FIG. 2, the user may provide input to modify one or more components of a preset configuration. Additionally, while the example embodiment displayed in FIG. 1 shows the integrated online system 105 as a separate server communicatively coupled to the client device 115 and the integrated stationary unit 125 through the network 110, in other example embodiments, the integrated online system 105 is integrated into the client device 115 and/or the integrated stationary unit 125.

The network 110 may comprise any combination of local area and/or wide area networks, using both wired and/or wireless communication systems. In one embodiment, the network 110 uses standard communications technologies and/or protocols. For example, the network 110 includes communication links using technologies such as Ethernet, 802.11, worldwide interoperability for microwave access (WiMAX), 3G, 4G, code division multiple access (CDMA), digital subscriber line (DSL), etc. Examples of networking protocols used for communicating via the network 110 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over the network 110 may be represented using any suitable format, such as hypertext markup language (HTML) or extensible markup language (XML). Those skilled in the art will recognize that encryption using other suitable techniques will be appropriate for various applications based on the nature of the network 110.

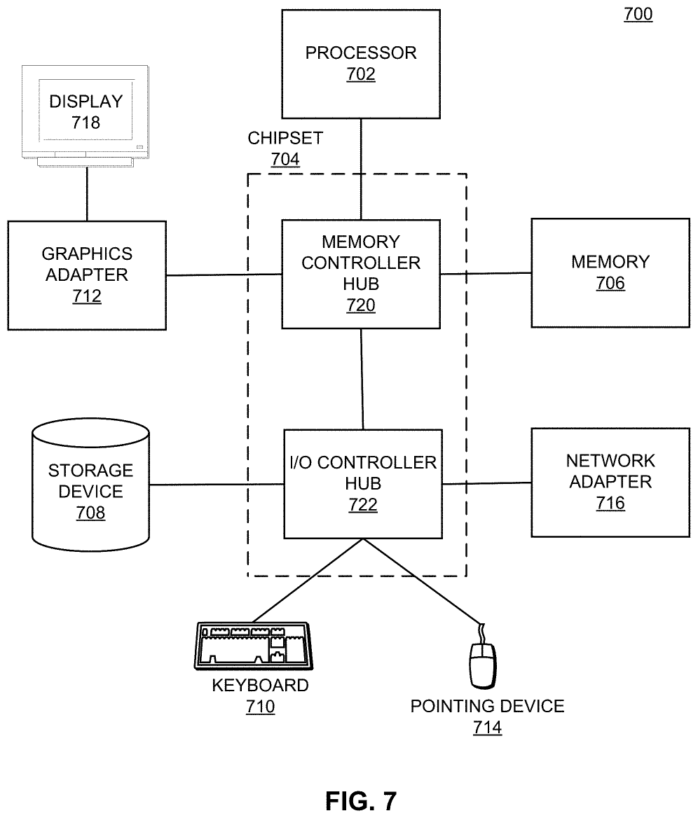

The client device 115 is a computing device capable of receiving user input as well as transmitting and/or receiving data via the network 110. In one embodiment, the client device 115 is a conventional computer system, such as a desktop or laptop computer. Alternatively, the client device 115 is a device having computer functionality, such as a mobile telephone, a smartphone, a set-top box, a smart home device, or another suitable device. The client device 115 further includes a camera capable of capturing images and videos, an input/output (I/O) component to transfer data to, and receive data from, other entities in the integrated wellness system 100, and a storage unit to store, for example, user-specific mappings generated by the integrated online system 105. An example of some or all components of an architecture of the client device 115 is illustrated and described with respect to FIG. 7.

The client device 115 executes an application, such as a wellness application 120 associated with the integrated online system 105. In one embodiment, the wellness application 120 includes a user interface that provides for display sensor data received from the integrated stationary unit 125, user-specific wellness mappings generated by the integrated online system 105, personalized configurations of the integrated stationary units 125, and user-specific wellness recommendations. The wellness application 120 is further capable of receiving user input, such as an instruction to modify a physical configuration of an integrated stationary unit 125 or to generate or adjust a user-specific mapping.

The integrated stationary unit 125 is an item of furniture configured to support the bodies of one or more individuals sitting, laying, or otherwise putting weight on the integrated stationary unit 125. Exemplary integrated stationary units 125 include, but are not limited to, couches, beds, daybeds, reclining chairs, lounge chairs, desk chairs, and the like. In one embodiment, one or more components of the integrated stationary unit 125, such as the arm rests, back support, seat slope, surface contours, or foot support, can be automatically or manually adjusted to different positions or angles to provide differing amounts of support to the individual. One or more servo or actuary motors are connected to hinge assemblies in the integrated stationary unit 125 to control the adjustment of the one or more components. Additionally, in some embodiments, the integrated online system 105 stores one or more preset configurations of the components of the integrated stationary unit 125. For example, a preset “reading” configuration might include a back component configured at a 90-degree angle, a seat component configured at a 20-degree angle, and a foot support component configured at a 0-degree angle.

The integrated stationary unit 125 includes one or more biometric sensors 130 placed at predefined locations in the integrated stationary unit 125. The biometric sensors 130 are located at positions that capture data based on a plurality of different body types, heights, genders, and other factors and may be densely packed or spread out. In one embodiment, the biometric sensors 130 measure biometric data of users interacting with the integrated stationary unit 125 and report measured data to the integrated online system 105. Exemplary biometric sensors 130 include ballistocardiograph (BCG) sensors that measure user heart rate, lumbar, thoracic, and cervical reading sensors that measure pressure on regions of the spine, a pulse oximeter that measures an oxygen level in the blood, and electrocardiogram (ECG) sensors that measure electrical activity of the heart. The integrated stationary unit 125 further includes one or more pneumatic lifting/dropping balloons that are inflated or deflated by a coupled compression unit. The integrated online system 105 triangulates data collected from the sensors 130 with location data (e.g., GPS data received from a client device 115) to formulate patterns that help to identify a user interacting with the integrated stationary unit 125 without having to explicitly query a client device 115 to request the identity of the user. In some embodiments, the integrated online system 105 generates user-specific mappings and configures one or more components of the integrated stationary unit 125 based on the measured sensor data, as discussed in more detail below with respect to FIG. 2.

The integrated stationary unit 125 is an Internet of Things (IoT) device that connects to the network 110 to communicate with the integrated online system 105 and the client device 115. For example, the integrated stationary unit 125 can be configured to report (e.g., periodically or on-demand) one or more sensor readings from the biometric sensors 130 to the integrated online system 105 for purposes of generating a user-specific mapping or configuration. As another example, the integrated stationary unit 125 can be configured to receive user input via the application 120 to adjust one or more components (e.g., a back-support angle) of the integrated stationary unit 125. In some embodiments, the integrated stationary unit 125 further includes an on/off button, localized charging ports, USB ports, a wireless charging pad, and localized heating and cooling components.

Turning now to FIG. 2, it illustrates a system architecture of an integrated online system 105, according to one example embodiment. In the embodiment shown in FIG. 2, the integrated online system 105 includes a front-end module 205, a sensor data module 210, a mapping module 215, a unit configuration module 220, a model training module 225, and a user data store 225. In other embodiments, the integrated online system 105 includes additional, fewer, or different components for various applications. Conventional components, such as network interfaces, security functions, load balancers, failover servers, management and network operations consoles, and the like are not shown so as not to obscure the details of the system architecture. The modules and/or components of the integrated online system 105 may be embodied as program code (or software) and executable by a computing device. An example of some or all components of an architecture of the computing device that may execute the program code is illustrated and described with respect to FIG. 7.

The front-end module 205 facilitates communication between the client device 115 and the integrated online system 105. For example, in one embodiment, the user provides input through a client device 115 to register one or more integrated stationary units 125 and/or one or more users with the integrated wellness system 100 such that the integrated online system 105 generates one or more wellness mappings for the registered user(s) based on the sensor data received from the registered integrated stationary unit 125. The integrated online system 105 returns to the user, through the front-end module 205, data regarding the generated mappings and/or one or more preset configurations of an integrated stationary unit 125, and the user may further provide, through the client device 115, additional input to view or modify a mapping or preset configuration as well as restrictions regarding the storage and use of biometric sensor data associated with the user.

The sensor data module 210 receives data from the biometric sensors 130 in the integrated stationary unit 125. In one embodiment, a user provides input through the application 120 indicating that the user is sitting, lying on, or otherwise interacting with the integrated stationary unit 125 and wishes to share biometric sensor data with the integrated online system 105. Responsive to receiving the user input, the sensor data module 210 instructs a processor in the integrated stationary unit 125 to provide sensor data for use in generating a wellness mapping for the user. The biometric sensors 130 in the integrated stationary unit 125 return, to the sensor data module 210, sensor data including measurements calculated by the BCG sensors, the lumbar, thoracic, and cervical reading sensors, the pulse oximeter, the ECG sensors, and one or more additional sensors in the integrated stationary unit 125. In one embodiment, sensor data is transmitted periodically from the biometric sensors 130 and may continually be transmitted to the sensor data module 210 until the user provides input including an instruction to stop data transmission or the sensor data indicates that the user is no longer interacting with the integrated stationary unit 125. In one example embodiment, the rate at which sensor data is collected and transmitted is based in part on a charge level of an associated battery or other charging mechanism. For instance, if the integrated stationary unit 125 is powered by a fully charged battery or connected outlet, the data capture frequency is approximately 60 seconds, and the data is transmitted to the sensor data module 210 approximately every 300 seconds. In instances where the integrated stationary unit 125 is not connected to an outlet and the battery level has decreased, the frequency of data collection and transmission is reduced to a minimum level to preserve local battery operations. Additionally, in some embodiments, the sensor data is captured and analyzed locally at the integrated stationary unit 125.

In some embodiments, the sensor data module 210 instructs the integrated stationary unit 125 to return sensor data measurements associated with a user of the integrated wellness system 100 without receiving user input comprising an instruction to begin data transmission. For example, responsive to detecting that a user is interacting with the integrated stationary unit 125 (e.g., responsive to receiving sensor data indicating that a user is sitting or lying on the integrated stationary unit 125), the sensor data module 210 compares the received sensor data to stored sensor data and previously generated user-specific wellness mappings. If a comparison between the received data and the stored sensor data and/or wellness mappings exceeds a similarity threshold, the sensor data module 210 determines that the user associated with the stored data and/or the wellness mapping is currently interacting with the integrated stationary unit 125 and instructs the integrated stationary unit 125 to continue to transmit sensor data for the identified user. In some embodiments, the sensor data module 210 further queries a client device 115 associated with the identified user to confirm that the user is currently interacting with the integrated stationary unit 125 (e.g., “Are you sitting on the living room sofa?”). Additionally or alternatively, during a registration process, an owner of the integrated stationary unit 125 may identify, through the application 120, one or more users who intend to interact with the integrated stationary unit 125 and provide identifying characteristics for each user, such as the user's height, weight, and the like. When a user interacts with the integrated stationary unit 125, the sensor data module 210 receives biometric sensor data and compares the received data to the identifying characteristics to determine an identity of the interacting user. In other embodiments, software on the integrated stationary unit 125 is used to map the sitting data pattern to its closest user match and determine the user identity.

The sensor data module 210 transmits the received sensor data to the mapping module 215 for use in generating the user-specific mapping and to the user data store 245 for storage in a user profile. In one embodiment, storage and use of the sensor data associated with users of the integrated wellness system 100 is subject to user permissions. For example, user permission input may dictate that the integrated online system 105 may store sensor data measurements for a specified period of time or may share gathered sensor data with one or more third-party systems (e.g., a client device 115 associated with a user's doctor or physical therapist). Additionally, in some embodiments, the sensor data module 210 encrypts the user sensor data prior to data storage and/or transmission.

The mapping module 215 receives sensor data measurements from the sensor data module 210, along with an identification of a user with whom the measured data is associated, and uses the received data to generate or augment a wellness mapping for the identified user. In one embodiment, the wellness mapping comprises a graph, chart, or other data visualization that includes historical sensor data for the user, including indications of when sensor data was received, an identification of the integrated stationary unit 125 and the sensor(s) in the unit that transmitted the data, and one or more data measurements. Additionally, the mapping module 215 may compute or identify one or more wellness statistics using the received data, such as a mean, median, or mode of data received from a specified sensor, an identification of a highest or a lowest measurement received from the sensor along with an indication of when the highest or lowest measurement was received, or an outlier measurement that varies from a mean sensor measurement for the user by more than a threshold amount. Still further, a user-specific mapping may include other demographic and health data for the user, including a user height, weight, or identification of relevant medical conditions, such as sciatica, a herniated disc, muscle strain, a degenerative disc disease, arthritis, and the like.

The mapping module 215 sends the generated or augmented wellness mapping to the user for display through the application 120 and, in some embodiments, receives user input via the front-end module 120 regarding the use or storage of the wellness mapping. For example, the user might provide an instruction to send a copy of, or a link to, the generated mapping to a client device 115 associated with the user's doctor. User permissions associated with the data sharing request may limit the third-party's access to the mapping and data to a specified time period or may allow the third-party to access only some of the measured data. Additionally, the user may provide input to modify one or more components of the mapping, for example, by disclosing a medical condition or a recent injury.

The model training module 220 applies machine learning techniques to generate a configuration model that uses the sensor data measurements and mappings to compute user-specific recommended configurations of one or more integrated stationary units 125 and to generate posture recommendations and feedback to users of the integrated online system 105. As part of the generation of the configuration model, the model training module 220 forms training data by identifying a training set of user sensor measurements and associated configurations of integrated stationary units 125. The model training module 220 extracts feature values from the training data, the features being variables deemed potentially relevant to the association of one or more sensor data measurements and one or more component configurations of the integrated stationary units 125. Specifically, the features extracted by the model training module 220 include sensor data measurements received from the biometric sensors 130, a configuration of one or more components of the integrated stationary unit 125 when the sensor data measurements were taken, a time when the sensor data measurements were taken, an amount of time that the user remains on or otherwise interacting with the integrated stationary unit 125, relevant medical conditions of the user, and the like.

The model training module 220 uses supervised machine learning to train the configuration model, with the feature vectors of the training data serving as the inputs. Different machine learning techniques—such as linear support vector machine (linear SVM), boosting for other algorithms (e.g., AdaBoost), neural networks, logistic regression, naïve Bayes, memory-based learning, random forests, bagged trees, decision trees, boosted trees, or boosted stumps—may be used in different embodiments. The configuration model, when applied to feature vectors extracted from the received sensor data and/or user-specific wellness mapping, outputs a recommended optimal configuration of one or more integrated stationary units 125 to improve the user's posture, comfort, and spinal health. In embodiments where the configuration model has previously generated a recommended configuration of an integrated stationary unit 125 for a user, additional user data transmitted by the biometric sensors 130 and/or an updated wellness mapping for the user are used as input to the trained model, which may output an updated recommended configuration based on the additional sensor data. Still further, in some embodiments, the configuration model further outputs one or more wellness or lifestyle recommendations associated with the recommended configuration. For example, the model might output a recommendation that the user sit all the way back in her desk chair or place her feet flat on the ground to improve posture or provide recommended stretches for the user based on the wellness mapping.

The recommended configuration of the integrated stationary unit 125 and, optionally, the wellness recommendations are output to the user through the front-end module 205. In one embodiment, one or more components of the recommended configuration may be modified based on user input. For example, a recommended optimal configuration for a user using a laptop on the integrated stationary unit 125 might include a back support configured at an 80-degree angle and a seat configured at a 30-degree angle. However, the user might provide input that overrides the configuration generated by the model, for instance, by adjusting the back-support to a 70-degree angle.

The unit configuration module 220 instructs the integrated stationary unit 125 to adjust one or more components of the unit 125 based on the recommended optimal configuration and/or user-specified manual adjustments. For example, responsive to the user providing input through the application 120 indicating that the user is sitting, lying on, or otherwise interacting with the integrated stationary unit 125 (or, alternatively, responsive to the sensor data module 210 detecting that the user is interacting with the integrated stationary unit 125), the unit configuration module 220 retrieves the applicable wellness mapping for the user from the user data store 245 and instructs the integrated stationary unit 125 to configure one or more components according to the mapping. For example, the unit configuration module 220 might instruct the integrated stationary unit 125 to adjust a back support to an approximately 140-degree angle computed by the configuration model as an optimal back support angle for the user.

The integrated stationary unit 125 notifies the unit configuration module 220 when the components of the integrated stationary unit 125 have been adjusted to the recommended configuration for the user. In one embodiment, the sensor data module 210 continues to monitor sensor data received from the biometric sensors 130 and sends the additional sensor data to the configuration model, which outputs, in some embodiments, further modifications of the integrated stationary unit 125 based on the additional sensor data. Alternatively, the configuration of one or more components of the integrated stationary unit 125 may further be adjusted based on user input through the application 120.

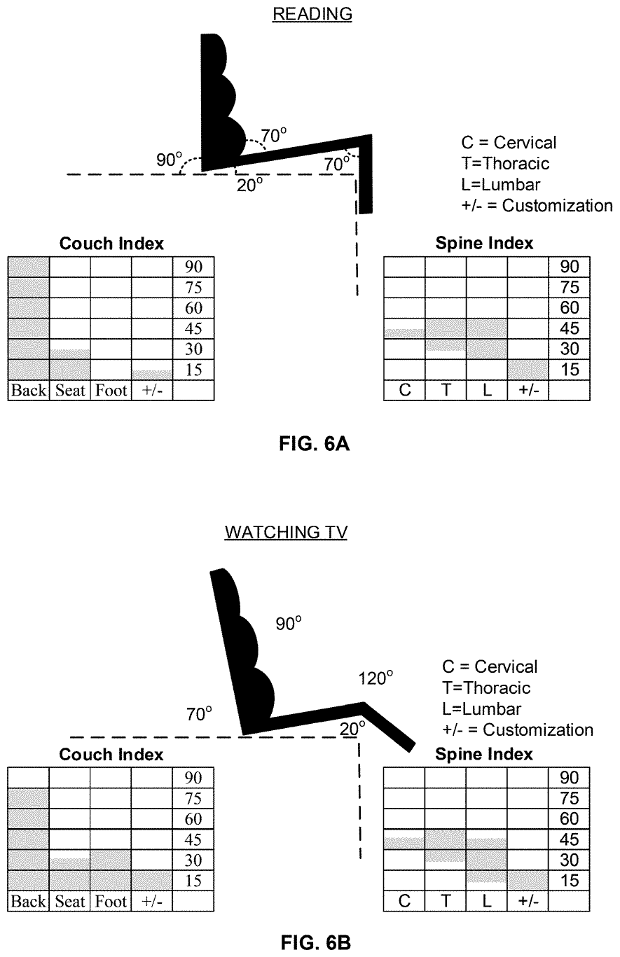

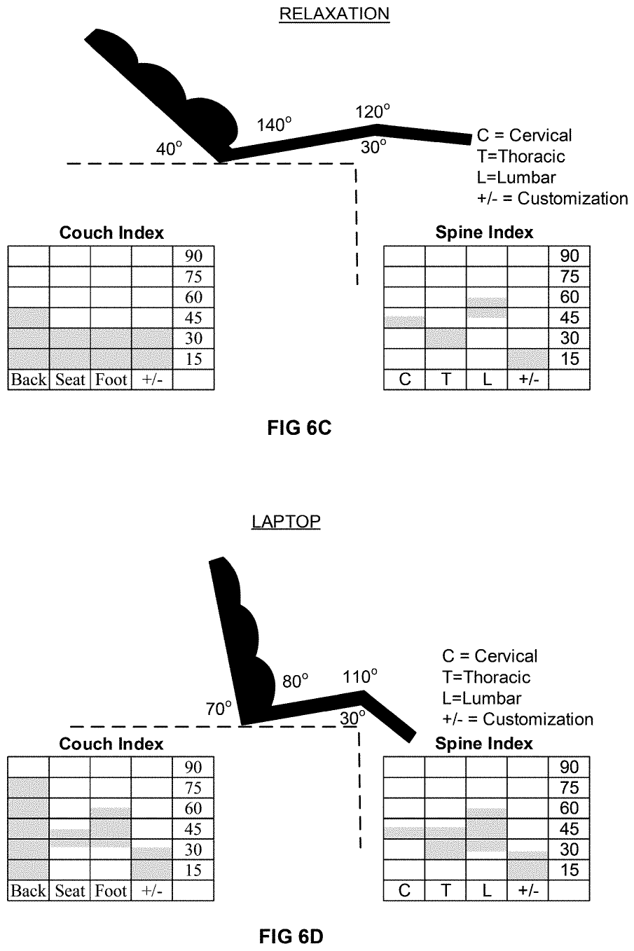

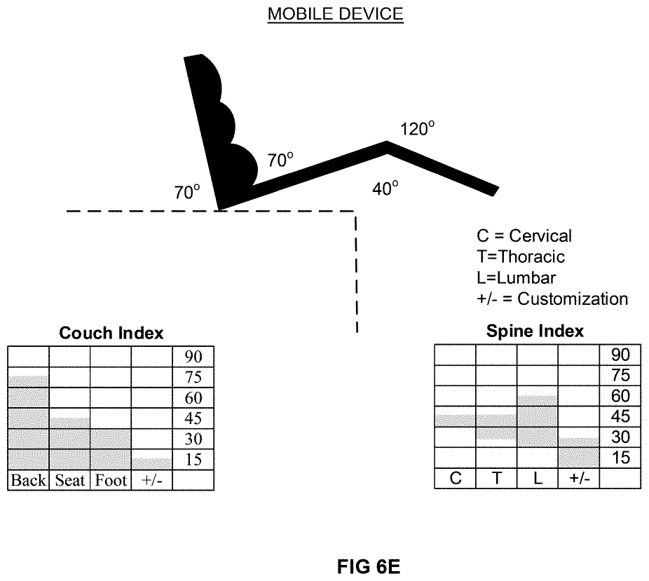

In some embodiments, the unit configuration module 220 generates one or more preset configurations of the integrated stationary unit 125 that may be used regardless of whether an individual interacting with the integrated stationary unit 125 is a user of the integrated wellness system 100. That is, in some embodiments, the one or more preset configurations are not based on sensor data associated with a specific user, but are instead based on recommended configurations generated by the unit configuration module 220 for one or more designated activities. For example, as discussed in more detail below with respect to FIGS. 6A-6C, the preset configurations may include configurations associated with a “reading” position, a “watching television” position, a “relaxation” position, a “laptop” position, a “mobile device” position, and the like. Additionally, users of the integrated stationary unit 125 may add one or more additional preset configurations by providing input through the application 120 to record a specified configuration that was adjusted manually. The additional preset configuration is stored in association with the specified user such that the user may later provide input to automatically adjust the integrated stationary unit 125 to the additional preset configuration without the need for further manual adjustment.

Each preset configuration of the integrated stationary unit 125 is associated with one or more indices representing measurements associated with the preset configuration. For example, in one embodiment, a first index includes a recommended angle or position of each component of the integrated stationary unit 125 as well as an indication of whether a user of the integrated wellness system 100 has provided input to customize the recommended angles, and if so, the amount of customization. The first index further includes an adjustment factor that may be based on a user's specific body type or medical condition and that may be entered manually by the user or inferred through model variables to account for the various body types of users interacting with the integrated stationary unit 125. A second index includes measures an amount of pressure on different portions of the user's spine.

The user data store 225 stores user profiles associated with each user of the integrated online system 105. A user profile includes declarative information about the user that was explicitly shared by the user and may also include profile information inferred by the integrated online system 105. In one embodiment, a user profile includes multiple data fields, each describing one or more attributes of the corresponding user of the integrated online system 105. Examples of information stored in a user profile include biographic, demographic (e.g., age or gender), medical (e.g., height, weight, known medical conditions), sensor readings from the biometric sensors 130 in one or more integrated stationary units 125, and the like. In one embodiment, the user profile further includes one or more user-specific mappings and preset configurations for one or more integrated stationary units 125 associated with the user.

### Example Integrated Stationary Unit

FIG. 3 illustrates an exemplary integrated stationary unit 125 having a plurality of biometric sensors 130. In the example embodiment shown in FIG. 3, the integrated stationary unit 125 is a couch. However, in other embodiments, the integrated stationary unit 125 is, for example, a bed, a daybed, a reclining chair, a lounge chair, a desk chair, or other sitting assembly.

The integrated stationary unit 125 is configurable such that one or more components of the integrated stationary unit 125 may be adjusted manually based on user input through the client device 115 or automatically based on a user-specific wellness mapping or preset configuration. In one embodiment, the integrated stationary unit 125 includes one or more motor hinge assemblies 305 that control the angle of each adjustable component. For example, in an embodiment where the integrated stationary unit 125 is a couch, the integrated stationary unit 125 includes three motor hinge assembles: a first assembly 305A located at a first end of a horizontal portion of the couch, a second assembly 305B located at a second end of the horizontal portion of the couch, and a third assembly 305C located at a base of a vertical portion of the couch and spanning the length of the horizontal portion of the couch. The first and second assemblies 305A and 305B can thus be used to control an angle of a headrest and/or a footrest if the user is lying on the integrated stationary unit 125, and the third assembly 305C can be used to control a back-support angle if the user is sitting up on the integrated stationary unit 125.

The integrated stationary unit 125 includes a plurality of biometric sensors 130 located at predefined locations in the interior of the integrated stationary unit 125. For example, in one embodiment, a plurality of cervical reading sensors 310 are configured in rows spanning a horizontal length of a vertical portion of the integrated stationary unit 125 to measure an amount of pressure on the top of a user's spine. Similarly, a plurality of thoracic reading sensors 315 are configured in rows and positioned below the cervical reading sensors 310 to measure an amount of pressure on the midpoint of the user's spine, and a plurality of lumbar reading sensors 320 are configured in rows and positioned below the thoracic reading sensors 315 at a base of the vertical portion of the integrated stationary unit 125 to measure an amount of pressure on the base of the user's spine. The biometric sensors 130 include both input sensors that collect data (e.g., force-sensitive resistors) and output sensors comprising actuators that control pneumatic lifting and dropping balloons in the integrated stationary unit 125 for pressure stabilization or angle adjustment.

The pneumatic lifting and dropping balloons are located at predefined locations in the interior of the integrated stationary unit 125. In one example embodiment, the balloons include a cervical balloon 325, a thoracic balloon 330, and an inflatable bladder (e.g., a lumbar balloon) 335. The inflatable bladders (e.g., lumbar balloons) 335 are controlled by an actuator in the integrated stationary unit 125 that receives a signal from a software algorithm running on the integrated online system 105 or the integrated stationary unit 125.

### Example Model Generation and Training Method

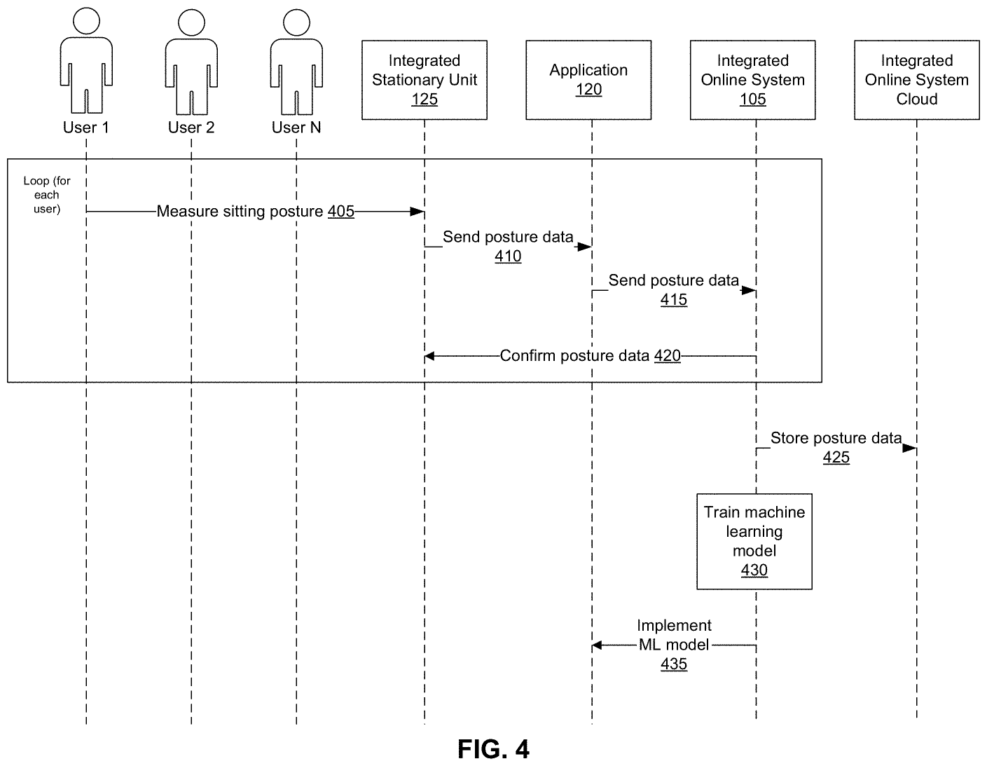

FIG. 4 illustrates a method 400 for training a configuration model to provide user-specific configurations of the integrated stationary unit 125 and feedback on postures and corrective measures to users of the integrated wellness system 100, according to one embodiment. The steps of FIG. 4 are illustrated from the perspective of the integrated online system 105 performing the method 400. However, some or all of the steps may be performed by other entities or components. In addition, some embodiments may perform the steps in parallel, perform the steps in different orders, or perform different steps.

The model training module 225 trains the configuration model using training data for each user (one, two, or more) of the integrated online system 105. To generate the training data, the integrated stationary unit 125 measures 405, for each user included in the training data set, posture data received from the biometric sensors 130 in the integrated stationary unit 125. In one embodiment, the measured posture data includes sensor data received from the lumbar, thoracic, and cervical reading sensors, the measurements representing an amount of pressure on different portions of the user's spine.

At 410, the integrated stationary unit 125 sends the posture data for the subject user to the application 120. In one embodiment, the application 120 interacts with the integrated stationary unit 125 to receive user input and provide to the user notifications regarding collected data and associated feedback and recommended corrective measures.

The application 120 sends (or transmits) 415 the posture data for the subject user to the integrated online system 105 for use in training a configuration model to generate user-specific configurations of the integrated stationary unit 125 and to provide feedback and corrective measures to users through the application 120. In one embodiment, the model training module 225 confirms 420 the posture data with the integrated stationary unit 125 before using the received data as training input to the configuration model.

The integrated online system 105 stores 425 the posture data for each user in the training set. In one embodiment, the model training module 225 sends the received posture data to an integrated online system cloud for storage. Alternatively, the data is stored in a user profile for each subject user in the user data store 245 on the integrated online system 105.

The model training module 225 on the integrated online system 105 uses the posture data for users in the training data set to train 430 the configuration model. As discussed above with respect to FIG. 2, the model training module 225 extracts feature values from the training data and uses the extracted feature values as inputs to the configuration model. Different machine learning techniques—such as linear SVM, boosting for other algorithms (e.g., AdaBoost), neural networks, logistic regression, naïve Bayes, memory-based learning, random forests, bagged trees, decision trees, boosted trees, or boosted stumps—may be used in different embodiments. After training the model, the integrated online system 105 implements 435 the trained model to the application 120. The trained model is configured to output, for a given set of posture data or other set of sensor data received from the integrated stationary unit 125 for a user of the integrated online system 105, a recommended configuration of the integrated stationary unit 125 and/or one or more recommendations to the user regarding posture and corrective measures. For example, the model might output a recommendation that the user stand or stretch if the sensor data indicates that the user has been sitting or lying on the integrated stationary unit 125 for at least a threshold amount of time or might recommend that the user watch a specified video based on the user's posture condition. Additionally, in some embodiments, the recommendations include one or more photos associated with the recommended corrective measures.

### Example Wellness Mapping and Adjustment Method

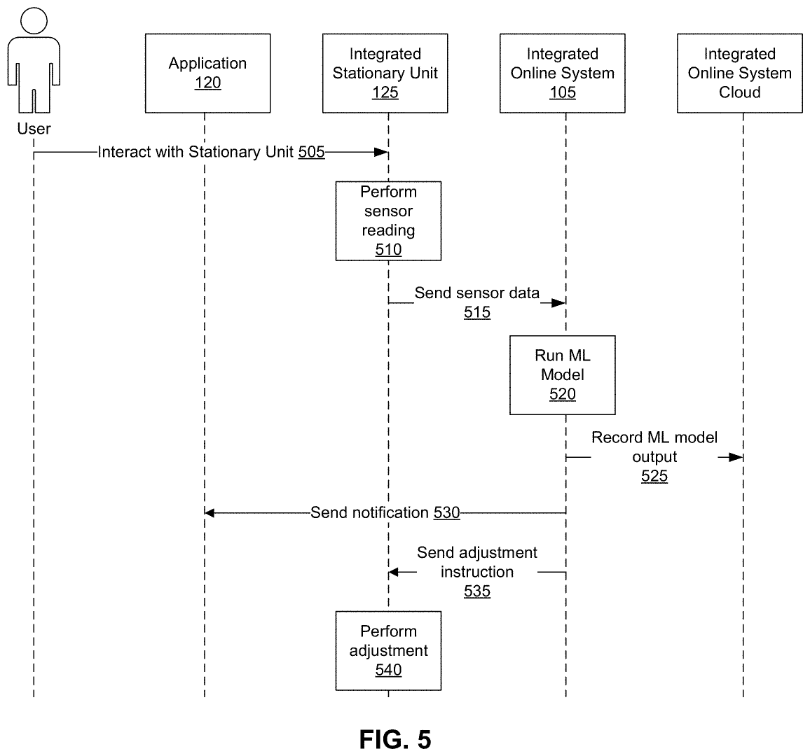

FIG. 5 illustrates an example process (or method) 500 for using the trained configuration model to generate a user-specific configuration of the integrated stationary unit, according to one embodiment. The steps of FIG. 5 are illustrated from the perspective of the integrated online system 105 performing the method 500. However, some or all of the steps may be performed by other entities or components. In addition, some embodiments may perform the steps in parallel, perform the steps in different orders, or perform different steps.

A user of the integrated online system 105 interacts 505 with the integrated stationary unit 125 by sitting on, laying on, or otherwise resting on the integrated stationary unit 125. In one embodiment, the user provides input through the application 120 to notify the integrated online system 105 that she is interacting with the integrated stationary unit 125. Alternatively, the integrated online system 105 queries the client device 115 to determine whether the user is interacting with the integrated stationary unit 125 responsive to receiving sensor readings from the integrated stationary unit 125.

The integrated stationary unit 125 performs 510 sensor readings based on the user interaction with the integrated stationary unit 125. For example, as discussed above with respect to FIG. 3, biometric sensors 130 positioned at predefined locations within the integrated stationary unit 125 include cervical reading sensors 310, thoracic reading sensors 315, and lumbar reading sensors 320. Additional sensors positioned in the integrated stationary unit 125 include, in various embodiments, BCG sensors, a pulse oximeter, and ECG sensors.

The integrated stationary unit 125 sends 515 the sensor data from the biometric sensors 130 to the integrated online system 105 for use in generating or updating user-specific wellness mappings and generating one or more recommended configurations of the integrated stationary unit 125. In one embodiment, sensor data is transmitted periodically (e.g., every 60 seconds) from the biometric sensors 130 and may continually be transmitted to the integrated online system 105 until the user provides input including an instruction to stop data transmission or the sensor data indicates that the user is no longer interacting with the integrated stationary unit 125. In other embodiments, the sensor data is transmitted once (e.g., after the user has been interacting with the integrated stationary unit 125 for a predefined period of time) or a predefined number of times.

At 520, modules of the integrated online system 105 run the configuration model on the sensor data received from the integrated stationary unit 125 and the configuration model outputs, based on the received sensor data, a recommended configuration of one or more components of the integrated stationary unit 125. For example, a recommended configuration might include a back support configured at a 90-degree angle, a seat configured at a 20-degree angle, and a foot support configured at a 0-degree angle and include a recommendation that the user sit upright in the integrated stationary unit 125 to minimize strain on the user's back. In some embodiments, the sensor data and/or the recommended configuration are additionally used to generate or update a user-specific wellness mapping for the user of the integrated online system 105.

The integrated online system 105 records 525 the configuration and recommendation output from the configuration model in the integrated online system cloud and, optionally, in the user data store 245 on the integrated online system 105. At 530, the integrated online system 105 sends a notification to the user through the application 120 with a recommended configuration of the integrated stationary unit 125 and, optionally, one or more recommendations regarding posture and corrective measures based on the sensor data measurements and output of the configuration model. In one embodiment, the notification includes a query asking the user to confirm that the integrated stationary unit 125 should be configured according to the recommended configuration. In various embodiments, the user may provide input through the application 120 to confirm the recommended configuration or to modify one or more components of the integrated stationary unit 125 (e.g., by changing the configuration of the foot support from the recommended 0-degree angle to a 30-degree angle).

Responsive to receiving confirmation or modification of the recommended configuration through the client device 115, the integrated online system 105 sends 535 an adjustment instruction to the integrated stationary unit 125 and the integrated stationary unit 125 performs 540 the requested adjustment. The adjusted configuration may be used to update a user-specific wellness mapping and/or a user profile for the user. Additionally, in some embodiments, the user may provide further input through the application 120 to make additional modifications to one or more components of the integrated stationary unit 125 after the integrated stationary unit has reached the requested configuration.

### Example Preset Configurations of Stationary Unit

FIGS. 6A-6C illustrate example preset configurations of the integrated stationary unit 125. As discussed above with respect to FIG. 2, the unit configuration module 220 generates one or more preset configurations for activities frequently associated with the integrated stationary unit. A user may provide input through the application 120 to instruct the integrated online system 105 to configure the integrated stationary unit 125 to one of the preset configurations or to further modify one or more components of the integrated stationary unit 125.

FIG. 6A illustrates a preset configuration 605A and indices 610A and 615A associated with a “reading” configuration of the integrated stationary unit 125. In the embodiment shown in FIG. 6A, the reading configuration of the integrated stationary unit 125 includes a back support configured at a 90-degree angle, a seat configured at a 20-degree angle, and a foot support configured at a 0-degree angle. The integrated stationary unit index 610A includes angle measurements for each of the components in the reading configuration as well as an indication of an amount of customization of one or more components of the integrated stationary unit 125. For instance, in the displayed embodiment, the customized portion is the seat and the angle between the horizontal and vertical assemblies. Additionally, the spine index 615A provides a measurement scale that indicates the amount of pressure on portions of the user's spine.

FIG. 6B illustrates a preset configuration 605B and indices 610B and 615B associated with a “watching TV” configuration of the integrated stationary unit 125. In the embodiment shown in FIG. 6B, the watching TV configuration of the integrated stationary unit 125 includes a back support configured at a 70-degree angle, a seat configured at a 20-degree angle, and a foot support configured at a 40-degree angle. The integrated stationary unit index 610B includes angle measurements for each of the components in the reading configuration as well as an indication of an amount of customization of one or more components of the integrated stationary unit 125. Additionally, the spine index 615B provides a measurement scale that indicates the amount of pressure on portions of the user's spine.

FIG. 6C illustrates a preset configuration 605C and indices 610C and 615C associated with a “relaxation” configuration of the integrated stationary unit 125. In the embodiment shown in FIG. 6C, the relaxation configuration of the integrated stationary unit 125 includes a back support configured at a 40-degree angle, a seat configured at a 30-degree angle, and a foot support configured at a −10-degree angle. The integrated stationary unit index 610C includes angle measurements for each of the components in the relaxation configuration as well as an indication of an amount of customization of one or more components of the integrated stationary unit 125. Additionally, the spine index 615C provides a measurement scale that indicates the amount of pressure on portions of the user's spine.

FIG. 6D illustrates a preset configuration 605D and indices 610D and 615D associated with a “laptop” configuration of the integrated stationary unit 125. In the embodiment shown in FIG. 6C, the laptop configuration of the integrated stationary unit 125 includes a back support configured at a 70-degree angle, a seat configured at a 30-degree angle, and a foot support configured at a 40-degree angle. The integrated stationary unit index 610D includes angle measurements for each of the components in the laptop configuration as well as an indication of an amount of customization of one or more components of the integrated stationary unit 125. Additionally, the spine index 615D provides a measurement scale that indicates the amount of pressure on portions of the user's spine.

FIG. 6E illustrates a preset configuration 605E and indices 610E and 615E associated with a “mobile device” configuration of the integrated stationary unit 125. In the embodiment shown in FIG. 6E, the mobile device configuration of the integrated stationary unit 125 includes a back support configured at a 70-degree angle, a seat configured at a 40-degree angle, and a foot support configured at a 20-degree angle. The integrated stationary unit index 610E includes angle measurements for each of the components in the mobile device configuration as well as an indication of an amount of customization of one or more components of the integrated stationary unit 125. Additionally, the spine index 615E provides a measurement scale that indicates the amount of pressure on portions of the user's spine.

### Computing System Architecture

FIG. 7 is a block diagram illustrating physical components of an example computer 700. All or parts of the components of example computer 700 may be configured for the integrated online system 105, in accordance with an embodiment. Illustrated are at least one processor 702 coupled to a chipset 704. Also coupled to the chipset 704 are a memory 706, a storage device 708, a graphics adapter 712, and a network adapter 716. A display 718 is coupled to the graphics adapter 712. In one embodiment, the functionality of the chipset 704 is provided by a memory controller hub 720 and an I/O controller hub 722. In another embodiment, the memory 706 is coupled directly to the processor 702 instead of the chipset 704.

The storage device 708 is any non-transitory computer-readable storage medium, such as a hard drive, compact disk read-only memory (CD-ROM), DVD, or a solid-state memory device. The memory 706 holds instructions and data used by the processor 702. The graphics adapter 712 displays images and other information on the display 718. The network adapter 716 couples the computer 700 to a local or wide area network.

As is known in the art, a computer 700 can have different and/or other components than those shown in FIG. 7. In addition, the computer 700 can lack certain illustrated components. In one embodiment, a computer 700, such as a host or smartphone, lacks a graphics adapter 712, and/or display 718, as well as a keyboard 710 or external pointing device 714. Moreover, the storage device 708 can be local and/or remote from the computer 700 (such as embodied within a storage area network (SAN)).

As is known in the art, the computer 700 is adapted to execute computer program modules for providing functionality described herein. As used herein, the term “module” refers to computer program logic utilized to provide the specified functionality. Thus, a module can be implemented in hardware, firmware, and/or software. In one embodiment, program modules are stored on the storage device 708, loaded into the memory 706, and executed by the processor 702.

### Additional Considerations

The disclosed configuration has benefits and advantages that include combining hardware and software components of the integrated wellness system 100 in a manner that reduces the user's risk of injury and improves overall well-being by making real-time recommendations and adjustments of the integrated stationary unit 125 based on biometric sensor data. Additionally, by storing sensor data and recommended configurations associated with a user, the system 100 can automatically adjust one or more components of the integrated stationary unit 125 responsive to determining that the user is interacting with the integrated stationary unit, thus obviating the need for repeated manual adjustments each time the user interacts with the integrated stationary unit 125.

Some portions of above description describe the embodiments in terms of algorithmic processes or operations. These algorithmic descriptions and representations are commonly used by those skilled in the data processing arts to convey the substance of their work effectively to others skilled in the art. These operations, while described functionally, computationally, or logically, are understood to be implemented by computer programs comprising instructions for execution by a processor or equivalent electrical circuits, microcode, or the like. Furthermore, it has also proven convenient at times, to refer to these arrangements of functional operations as modules, without loss of generality.

As used herein, any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. It should be understood that these terms are not intended as synonyms for each other. For example, some embodiments may be described using the term “connected” to indicate that two or more elements are in direct physical or electrical contact with each other. In another example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments. This is done merely for convenience and to give a general sense of the disclosure. This description should be read to include one or at least one and the singular also includes the plural unless it is obvious that it is meant otherwise.

Upon reading this disclosure, those of skill in the art will appreciate still additional alternative structural and functional designs for a system and a process for using biometric sensor data from a stationary unit to generate user-specific wellness mappings and to automatically make physical configuration adjustments of the stationary unit. Thus, while particular embodiments and applications have been illustrated and described, it is to be understood that the described subject matter is not limited to the precise construction and components disclosed herein and that various modifications, changes and variations which will be apparent to those skilled in the art may be made in the arrangement, operation and details of the method and apparatus disclosed. The scope of protection should be limited only by the following claims.

1. A computer-implemented method of automatically configuring an integrated stationary unit based on received biometric sensor data, the method comprising:
receiving, at an integrated online system, biometric sensor data from an integrated stationary unit having a plurality of biometric sensors;
identifying, using the received biometric sensor data, a user of the integrated online system currently interacting with the integrated stationary unit;
inputting the biometric sensor data for the identified user into a trained configuration model, the configuration model configured to generate, using the biometric sensor data, a recommended configuration of one or more components of the integrated stationary unit; and
transmitting, to the integrated stationary unit, an instruction to configure one or more components of the integrated stationary unit according to the recommended configuration.

2. The computer-implemented method of claim 1, further comprising:
receiving, from the integrated stationary unit, a notification that the one or more components of the integrated stationary unit have been configured according to the recommended configuration; and
providing for display on a client device associated with the identified user, a notification of the recommended configuration.

3. The computer-implemented method of claim 2, further comprising:
receiving, from the client device, user input comprising an instruction to modify the recommended configuration; and
transmitting, to the integrated stationary unit, an instruction to further modify one or more components of the integrated stationary unit based on the user input.

4. The computer-implemented method of claim 1, further comprising modifying, based on the received biometric sensor data, a wellness mapping associated with the identified user, the wellness mapping including a data visualization generated based on historical biometric sensor data associated with the identified user.

5. The computer-implemented method of claim 4, further comprising:
computing, based on the received biometric sensor data and the historical biometric sensor data associated with the identified user, one or more wellness statistics for the identified user;
generating, by the configuration model based on the computed wellness statistics, one or more wellness recommendations for the identified user; and
providing the one or more wellness recommendations for display on the client device.

6. The computer-implemented method of claim 1, wherein the plurality of biometric sensors include one or more of: ballistocardiograph (BCG) sensors, a pulse oximeter, and electrocardiogram (ECG) sensors.

7. The computer-implemented method of claim 1, further comprising:
receiving user input comprising an indication of a specified user activity; and
instructing the integrated stationary unit to adjust one or more components of the integrated stationary unit according to a preset configuration associated with the specified user activity.

8. An integrated wellness system, comprising:
an integrated stationary unit having a plurality of biometric sensors and configured to transmit biometric sensor data to an integrated online system responsive to determining that a user of the integrated online system is interacting with the integrated stationary unit;
the integrated online system communicatively coupled to the integrated stationary unit, the integrated online system configured to compute, based on the received biometric sensor data, a recommended configuration of the integrated stationary unit and to instruct the integrated stationary unit to adjust one or more components of the integrated stationary unit based on the recommended configuration; and
a wellness application executing on a client device and configured to display configuration information of the integrated stationary unit and to receive user input from the client device to further modify one or more components of the integrated stationary unit.

9. The integrated wellness system of claim 8, wherein the integrated online system is configured to generate the recommended configuration of the integrated stationary unit by inputting the received biometric sensor data into a trained configuration model.

10. The integrated wellness system of claim 8, wherein the plurality of biometric sensors include one or more of: ballistocardiograph (BCG) sensors, a pulse oximeter, and electrocardiogram (ECG) sensors.

11. The integrated wellness system of claim 8, wherein the integrated stationary unit is configured to periodically transmit biometric sensor data to the integrated online system, the integrated online system using the transmitted biometric sensor data to update a wellness mapping associated with the identified user.

12. The integrated wellness system of claim 11, wherein the wellness mapping includes a data visualization generated based on historical biometric sensor data associated with the identified user.

13. The integrated wellness system of claim 8, wherein the integrated online system is configured to identify a user interacting with the integrated stationary unit by comparing the received biometric sensor data with historical biometric sensor data associated with users of the integrated online system and the integrated stationary unit.

14. The integrated wellness system of claim 8, wherein the integrated online system is configured to identify a user interacting with the integrated stationary unit based on user input through the client device.

15. A non-transitory computer-readable storage medium configured to store instructions, the instructions when executed by a processor causing the processor to:
receive, at an integrated online system, biometric sensor data from an integrated stationary unit having a plurality of biometric sensors;
identify, using the received biometric sensor data, a user of the integrated online system currently interacting with the integrated stationary unit;
input the biometric sensor data for the identified user into a trained configuration model, the configuration model configured to generate, using the received biometric sensor data, a recommended configuration of one or more components of the integrated stationary unit; and
transmit, to the integrated stationary unit, an instruction to configure one or more components of the integrated stationary unit according to the recommended configuration.

16. The non-transitory computer-readable storage medium of claim 15, wherein the instructions further comprise instructions that, when executed, cause the processor to:
receive, from the integrated stationary unit, a notification that the one or more components of the integrated stationary unit have been configured according to the recommended configuration; and
provide for display on a client device associated with the identified user, a notification of the recommended configuration.

17. The non-transitory computer-readable storage medium of claim 16, wherein the instructions further comprise instructions that, when executed, cause the processor to:
receive, from the client device, user input comprising an instruction to modify the recommended configuration; and
transmit, to the integrated stationary unit, an instruction to further modify one or more components of the integrated stationary unit based on the user input.

18. The non-transitory computer-readable storage medium of claim 15, wherein the instructions further comprise instructions that, when executed, cause the processor to modify, based on the received sensor data, a wellness mapping associated with the identified user, the wellness mapping including a data visualization generated based on historical sensor data associated with the identified user.

19. The non-transitory computer-readable storage medium of claim 18, wherein the instructions further comprise instructions that, when executed, cause the processor to:
compute, based on the received sensor data and the historical sensor data associated with the identified user, one or more wellness statistics for the identified user;
generate, by the configuration model based on the computed wellness statistics, one or more wellness recommendations for the identified user; and
provide the one or more wellness recommendations for display on the client device.

20. The non-transitory computer-readable storage medium of claim 15, wherein the instructions further comprise instructions that, when executed, cause the processor to:
receive user input comprising an indication of a specified user activity; and
instruct the integrated stationary unit to adjust one or more components of the integrated stationary unit according to a preset configuration associated with the specified user activity.
